(19) United States
(12) Patent Application Publication
(10) Pub. No.: US 2005/0222608 A1
(43) Pub. Date: Oct. 6, 2005

(54) Acupressure device for treating insomnia

(76) Inventors: Wanzhu Hou, Rockville, MD (US); Guangpi Xu, Rockville, MD (US)

Correspondence Address: BINGHAM, MCCUTCHEN LLP, THREE EMBARCADERO CENTER, 18 FLOOR, SAN FRANCISCO, CA, 94111-4067, US

(21) Appl. No.: 10/813,057

(22) Filed: Mar. 31, 2004

(51) Int. Cl. A61B 17/00

(52) U.S. Cl. 606/204

(57) ABSTRACT

The present invention relates to an acupressure device for producing a calming/soothing effect in a person. The acupressure device applies pressure to a desired treatment zone and discharging the ionic imbalance of the treatment zone to producing such calming/soothing effect. In particular, the acupressure device is effective in treating a person having sleep problems, stress/tension, anxiety, depression, and/or imbalanced state of mine. The method of using the acupressure device for treatment of sleep problems, stress/tension, anxiety, depression, and/or imbalanced state of mind is described.

### FIELD OF INVENTION

The present invention relates to an acupressure device for producing a calming/soothing effect in a person. In particular, the acupressure device applies pressure to acupoint(s) on the head of a human to discharge ionic imbalance of the area, thus providing calming/soothing result in the human. The acupressure device is effective in treating a person with insomnia, stress/tension, anxiety, depression, and/or imbalanced state of mind.

### BACKGROUND OF THE INVENTION

Acupuncture is a kind of clinical practice which involves piercing specific peripheral nerves with needles to relieve the discomfort associated with painful disorders, to induce surgical anesthesia, and for therapeutic purposes. Acupuncture originated from China, which has a history that can be traced back to more than 2,500 years.

The concept behind acupuncture is based on the premise that there are patterns of energy flow (Qi) through the body that are essential for proper health. Disruptions of this flow are believed to be responsible for discomfort and disease. Correction of the energy flow imbalance to alleviate diseases and restore health may be achieved by acupuncture manipulation at identifiable points (i.e., acupoints) close to the skin. An acupoint is defined as a specific point for needle insertion along a body meridian in acupuncture.

Acupuncture manipulation includes a family of procedures involving stimulation of the identifiable points by a variety of manipulation or techniques. The best known method for stimulating acupuncture points employs the penetration of the skin by thin, solid, metallic needles, which can be manipulated manually or by electrical stimulation. Using needles of various types, the acupuncturist is able to reach these points by placing needles into the skin at these points. The insertion of a needle at an acupoint alters the flow of energy, changing the dynamics in the energy flow. When done properly, using the proper number and positions of needles, acupuncture re-aligns the energy flow to one of balance and restoration of health. Stimulation of these areas by moxibustion, pressure (also called acupressure), heat, and lasers are also used in acupuncture practice. (Acupuncture. NIH Consensus Statement Nov. 3-5, 1997; 15(5):1-34.)

As aforementioned, the pattern of energy flow disruption corresponds to the type of disease from which the patient is suffering. When treating a disease, the acupuncturist diagnoses the ailment complained of and then identifies the corresponding energy flow imbalance. The energy flow imbalance can be corrected by placing one or more needles into acupoints associated with the type of energy flow imbalance corresponding to the disease.

However, the sterility requirements of the acupuncture needles, the required skill levels of clinicians, and the need for office visits render the application of the acupuncture needles inconvenient. Alternatively, acupressure therapy (i.e., acupuncture using pressure stimulation instead of needles) has been used in replacement of acupuncture in certain conditions. Acupressure is the application of pressure (as with the thumbs or fingertips) to the same discrete points (i.e., the acupoints) on the body stimulated in acupuncture that is used for its therapeutic effects (as the relief of tension or pain). Acupressure applies a simple and inexpensive technique, with virtually no negative side effects. Acupressure could greatly enhance the treatment of many health conditions if it were readily available to the lay public.

There have been a few disclosures of acupressure devices in recent years. For example, U.S. Pat. No. 6,554,787 discloses a headband for treatment of headache with one or more pockets for the insertion of treatment elements including cold, heat, vibration, and magnetism. The headband is attached to the head in an annular fashion and tightening of the headband by pulling tight a pull-tab provides pressure to relieve headache.

U.S. Pat. No. 6,458,146 discloses an acupressure patch for use on a selected skin surface portion of a human body. The acupressure patch has a base sheet which has a margin. The margin has at least a portion of its length shaped as an orienting margin, which corresponds to the shape on or adjacent to a selected skin surface contour. The base sheet further contains protrusions and a fastener. The protrusions are arranged in a constellation. The fasteners includes magnetic fasteners (e.g., magnets), mechanical fasteners (e.g., a clamp, clip, wire, or a suction or spring loaded device), and chemical fasteners (e.g., a glue or adhesive).

U.S. Pat. No. 5,792,174 discloses a headache-relieving acupressure cap-like device having an outer membrane and an inner membrane. The device contains multiple plastic/rubber protrusions that are 10 to 20 mm apart and distributed over the entire area covered by the cap. The device is pressurized by pumping air into the space between the outer and inner membranes of the cap.

In the invention to be presented in the following sections, an acupressure device which has the function of calming/soothing humans and treatment of insomnia will be introduced. Briefly, the acupressure device has a supporting member. The supporting member is in the shape of a strip or headband, which can be tightened on the wearer's head. The device is particularly characterized by having metal membranes embedded in, affixed to and/or molded from the inner side of the supporting member. More specifically, the metal members are arranged based on the sites of the acupoints on the head, thereby, by tightening the supporting member, the metal members act as blunt needles which exert pressure to the acupoints on the head. The electrical conductivity of the metal members discharges the ionic imbalance of the body energy flow, which in turn sends a calming/soothing signal to the human body.

### SUMMARY OF THE INVENTION

Anatomical identification of acupoint has been well documented in the literature and meridian textbooks. For the convenience of reference, the acupoint in the present invention is referred to by its Chinese Pinyin name, its alphanumerical sequence in the meridian and the corresponding number in the figures. For example, Baihui (D20) acupoint (11) denotes the Baihui acupoint at the 20^{th} point in the Du meridian, which is shown as 11 in the figures. A table (i.e., Table 1) is further provided in the Detailed Description for acupoints referred to in the present invention.

In the first preferred embodiment of the present invention, an acupressure device is provided which contains a supporting member (1) having an inner side (2), and a metal member (3) attaching to the inner side (2) of the supporting member (1). The metal member may be embedded in, affixed to, and/or molded from the supporting member. The metal member (3) is positioned at Baihui (D20) acupoint (11) of a human located at top of a human head. The Baihui (D20) acupoint (11) can be identified by finding the intersection between a first line (i.e., a line drawn between the highest points of two upper ear curvatures) and a second line (i.e., the Du meridian, which is a meridian that starts from Changqiang (D1), passing through the middle of the head, to Yinjiao (D28) at the front of the head. The Baihui (D20) acupoint (11) is the slight indentation between the first line and second line. When the acupressure device is worn on the head of a wearer, due to tightening of the supporting member, the metal member (3) is pressed against the Baihui(D20) acupoint (11) of the human head, which in turn generated a tension on the Baihui (D20) acupoint to send a signal that calms and sooths the human.

The metal member (3) is preferably made of an electrically conductive metal. Examples of the metal include, but are not limited to, gold, silver, platinum, copper, iron, stainless steel, nickel, aluminum, tin, titanium, and any combination thereof with or without any alloys attainable with these metals. The most preferred metal is gold or silver. The metal member (3) is in the form of a bead, a round mass, a nubbin, a disk, a nipple, a nodule, a lump, a bump, a projection, a knob, or a seed, or any combination thereof.

The supporting member (1) is preferably made of a cloth (such as cotton, Dacron, polyester, or nylon), SPANDEX fabric, plastic, rubber, leather, metal, and/or any combination thereof. The supporting member (1) can be a strip or a headband. In the case of a strip, the supporting member (1) has two opposite ends (4, 4′) at a longitudinal side which can be directly fastened together or fastened together through a pair of fasteners (5, 5′). Examples of fasteners include, but are not limited to hooks, pull-tabs, buckles, snap-hooks, adjuster bars, slides, cord locks, zipper pulls, modular buckles and VELCROs. The fasteners are capable of being adjusted to tightness so as to exert pressure on the Baihui (D20) acupoint (11) on the human head.

The acupressure device can be used to treat a person with insomnia or stress/tension problem. It can also be worn to reduce anxiety/depression.

In the second preferred embodiment of the present invention, the acupressure device comprises a supporting member (1) having an inner side (2). A plurality of metal members (3′) attached to the inner side of the supporting member. The plurality of the metal members (3′) are positioned at a plurality of acupoints in a first treatment zone (201) on top of a human head. The first treatment zone is defined as a rectangular area extending from midpoints (70, 70′) of two upper eyelids to two Chengling (Gb18) acupoints (34, 34′) on the human head. One of the plurality of the acupoints is the Baihui (D20) acupoint (11). By way of tightening the supporting member (1), the plurality of the metal members (3′) are pressed against at least the Baihui (D20) acupoint (11) of the first treatment zone (201), which generate a tension toward the first treatment zone and in turn sends a signal that calms and sooths the human. Preferably, the plurality of the metal members (3′) further positioned at at least one acupoint which is selected from the group consisting of Yingtang acupoint (80), Shenting (D24) acupoint (15), Shangxing (D23) acupoint (14), Xinhui (D22) acupoint (13), Qianding (D21) acupoint (12), Houding (D19) acupoint (10), a pair of Zanzhu (B12) acupoints (20, 20′), a pair of Meichong (B13) acupoints (21, 21′), a pair of Quchai (B14) acupoints (22, 22′), a pair of Wuchu (B15) points (23, 23′), a pair of Chengguang (B16) acupoints (24, 24′), a pair Tongtian (B17) acupoints (25, 25′), a pair of Luoque (B18) acupoints (26, 26′), a pair of Yangbai (Gb14) acupoints (30, 30′), a pair of Head-Lingqi (Bb15) acupoints (31, 31′), a pair of Muchuang (Gb16) acupoints (32, 32′), a pair of Zhengying (Gb17) acupoints (33, 33′), and a pair of Chengling (Gb18) acupoints (34, 34′) of the head.

The first embodiment also provides a method for treating insomnia in a human by applying the aforementioned acupressure device to the human.

In the third preferred embodiment of the present invention, the acupressure device contains a supporting member (1) having an inner side (2) and a plurality of metal members (3) attaching to the inner side of the supporting member (1). The plurality of the metal members (3″) are positioned at a plurality of acupoints at a second treatment zone (202) on top of a human head. The second treatment zone (202) is defined by a rectangular area extending from the pair of Yangbai (Gb14) acupoints (30, 30′) to the pair Chengling (Gb18) acupoints (34, 34′) of the head. Baihui (D20) acupoint (11) is among the acupoints in the second treatement zone. By tightening the supporting member (1), the plurality of the metal members (3″) are pressed against the plurality of the acupoints at the second treatment zone (202) of the human head, which in turn generate a tension to send a signal that calms and sooths the human. Preferably, the multiple metal members (3″) are arranged in such a way that they are positioned at Shangxing (D24) acupoint (14), Xinhui (D22) acupoint (13), Qianding (D19) acupoint (12), a pair of Wuchu (B15) acupoints (23, 23′), a pair of Chengguang (B16) acupoints (24, 24′), a pair of Head-Lingqi (Gb15) acupoints (31, 31′), and a pair of Muchuang (Gb16) acupoints (32, 32′) of the human head. The third embodiment also provides a method for treating insomnia in a human by applying the aforementioned acupressure device to the human.

In the fourth preferred embodiment of the present invention, the acupressure device comprises a supporting member (1) having an inner side (2); and a plurality of metal members (3′″) attaching to the inner side (2) of the supporting member (1). The plurality of the metal members (3′″) are positioned at a plurality of acupoints at a third treatment zone (203) on top of a human head. The third treatment zone (203) is a rectangular area which is about 1 inch wide between Shenting (D24) acupoint (15) and Houding (D19) acupoint (10) of Du meridian (101). One of the plurality of the acupoints included in the third treatment zone is Baihui (D20)acupoint (11). By tightening the supporting member (1), the plurality of the metal members (3′″) are pressed against the third treatment zone (203) of the human head, which in turn generate a tension to send a signal that calms and sooths the human.

The fourth embodiment also provides a method for treating insomnia in a human by applying the aforementioned acupressure device to the human.

### BRIEF DESCRIPTION OF THE DRAWINGS

FIG. 1 illustrates Baihui (D20) acupoint (11) and the Du meridian (101): (a) a top view and (b) a rear view, of the human head showing the location of Baihui (D20)acupoint. Baihui (D20) acupoint is located at the top of the head, which is the intersection between the Du meridian and a line connecting the two upper curvatures of the ears.

FIG. 2 illustrates the area of the first treatment zone (201) on the head and the preferred acupoints in the first treatment zone: (a) a top view and (b) a rear view of the head showing the location of the acupoints. Dots represent the preferred acupoints. The first treatment zone (201) is a rectangular area defined by two midpoints (70, 70′) of the upper eyelids and two Chengling (Gb18) (34, 34′) acupoints of Gall Bladder meridian (111, 111′). The preferred acupoints within the first treatment zone include Yintang acupoint (80), Shenting(D24) acupoint (15), Shangxing (D23) acupoint (14), Xinhui (D22) acupoint (13), Qianding (D21) acupoint (12), Baihui (D20) acupoint (11), Houding (D19) acupoint (10), a pair of Zanzhu (B12) acupoints (20, 20′), a pair of Meichong (B13) acupoints (21, 21′), a pair of Quchai (B14) acupoints (22, 22′), a pair of Wuchu (B15) points (23, 23′), a pair of Chengguang (B16) acupoints (24, 24′), a pair of Tongtian (B17) acupoints (25, 25′), a pair of Luoque (B18) acupoints (26, 26′), a pair of Yangbai (Gb14) acupoints (30, 30′), a pair of Head-Lingqi (Gb15) acupoints (31, 31′), a pair of Muchuang (Gb16) acupoints (32, 32′), a pair of Zhengying (Gb17) acupoints (33, 33′), and a pair of Chengling (Gb18) acupoints (34, 34′).

FIG. 3 illustrates the second treatment zone (202) on the head and the preferred acupoints within the second treatment zone: (a) a top view, and (b) a rear view of the head showing the preferred acupoints. The second treatment zone (202) is a rectangular area defined by two Yangbai (Gb14) (30, 30′) acupoints and two Chengling (Gb18) (34, 34′) acupoints, both belong to Gall Bladder meridian (111, 111′). The preferred acupoints within the second treatment zone include Shangxing (D23) acupoint (14), Xinhui (D22) acupoint (13), Qianding (D21) acupoint (12), Baihui (D20) acupoint (11), Wuchu (B15) acupoints (23, 23′), Chengguang (B16) acupoints (24, 24′), Head-Lingqi (Gb15) acupoints (31, 31′), and Muchuang (Gb16) acupoints (32, 32′).

FIG. 4 illustrates the third treatment zone (203) on the head and the preferred acupoints within the third treatment zone: (a) a top view, and (b) the rear view of the head showing the preferred acupoints. The third treatment zone (203) is a rectangular area which is about 1 inch wide between Shenting acupoint (15) and Houding acupoint (10) of the Du meridian (101).

FIG. 5 illustrates various acupressure devices of the present invention: (a) an acupressure device with a headband supporting member (1) and a metal member (3); (b) an acupressure device with a headband supporting member (1) and a plurality of metal members (3′); (c) an acupressure device with a long-stripped supporting member (1), multiple metal members (3′″), and VELCRO fasteners (5, 5′) at two ends (4, 4′); and (d) an acupressure device with a cap supporting member (1) and a plurality of metal members (3′).

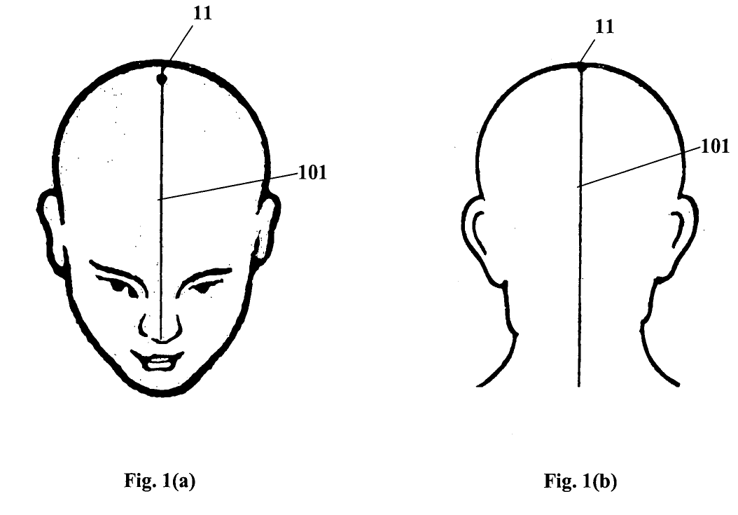

### DETAILED DESCRIPTION OF THE INVENTION

The energy flow patterns through the body have been mapped extensively throughout the human body, known as the meridian system. Along these energy flow courses (meridians), substantial numbers of acupoints at which acupuncture manipulation can bring on physiological responses and therapeutic effects have been identified. The majority of these acupoints are close to the skin surface. In addition to the term “acupoints” used throughout this invention, they can also be referred to as points, meridian points, acupuncture points, acupressure points, etc.

The courses of the meridians and the anatomical identification of the acupoints can be found in many literature and textbooks of acupuncture. For the convenience of referencing the acupoints, each acupoint described in the present invention is, as shown in Table 1, referred to by its Chinese Pinyin name, its alphanumerical sequence in the meridian and the corresponding number in the figures. For example, Baihui (D20) acupoint (11) denotes the Baihui acupoint at the 20^{th} point in the Du meridian, which is shown as 11 in the figures. The present invention adopts the Chinese Pinyin name of the meridians and acupoints in Li Ding et al., “Acupuncture, Meridian Theory and Acupuncture Points” (Beijing Foreign Languages Press, (1991), Beijing, China [www.MenDiseasesTCM.com/index.htm]).

TABLE 1
The Chinese Pinyin Name, Alphanumerical Sequence in the
Meridian and Corresponding Number in the Figures of the
Acupoints Referred in the Present Invention

| | | Alphanumerical | Corresponding |
|---|---|---|---|
| Corresponding | Chinese | Sequence in the | Number in the |
| Meridian | Pinyin name | Meridian | Figure |
| Du Meridian | Changqiang | D1 | not shown |
| (abbreviated as “D” in | Houding | D19 | 10 |
| the alphanumerical | Baihui | D20 | 11 |
| sequence) | Qianding | D21 | 12 |
| | Xinhui | D22 | 13 |
| | Shangxing | D23 | 14 |
| | Shenting | D24 | 15 |
| | Yingtang | not applicable | 80 |
| | Suliao | D25 | not shown |
| | Yinjiao | D28 | not shown |
| Foot-Taiyang Urinary | Jingmin | B11 | not shown |
| Bladder meridian | Zanhu | B12 | 20, 20′ |
| (abbreviated as “B1” | Meichong | B13 | 21, 21′ |
| in the alphanumeric | Quchai | B14 | 22, 22′ |
| sequence) | Wuchu | B15 | 23, 23′ |
| | Chengguang | B16 | 24, 24′ |
| | Tongtian | B17 | 25, 25′ |
| | Luoque | B18 | 26, 26′ |
| | Zhiyin | B167 | not shown |
| Foot-Shaoyang Gall | Tongziliao | Gb1 | not shown |
| Bladder meridian | Yangbai | Gb14 | 30, 30′ |
| (abbreviated as “Gb” | Head-Lingqi | Gb15 | 31, 31′ |
| in the alphanumeric | Muchuang | Gb16 | 32, 32′ |
| sequence) | Zhengying | Gb17 | 33, 33′ |
| | Chengling | Gb18 | 34, 34′ |
| | Foot-Qiaoyin | Gb44 | not shown |
| not applicable | midpoints of | not applicable | 70, 70′ |
| | the upper | | |
| | eyelids | | |

The meridian system consists of twelve regular meridians and eight extra meridians. The twelve Regular meridians are the: Hand-taiyin Lung meridian, Hand-shaoyin Heart meridian, Hand-jueyin Pericardium meridian, Hand-yangming Large Intestine meridian, Hand-taiyang Small Intestine meridian, Hand-shaoyan Sanjiao meridian, Foot-yangming Stomach meridian, Foot-taiyang Urinary Bladder meridian (110, 110′), Foot-shaoyang Gall Bladder meridian (111, 111′), Foot-taiyin Spleen meridian, Foot-Shaoyin Kidney meridian, and Foot-Jueyin Liver meridian. The eight Extra meridians are: Du (101), Ren, Chong, Dai, Yinqiao, Yangqiao, Yinwei and Yangwei meridians.

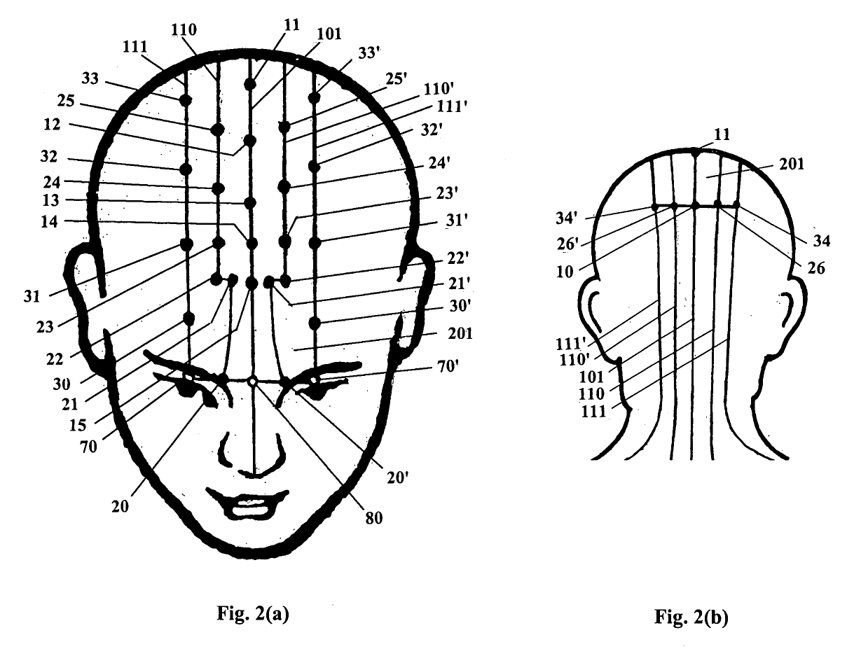

Among these meridians, the Du (101), Urinary Bladder (110, 110′), and Gall Bladder (111, 111′) meridians flow through the top of the head (FIG. 2), the Du meridian (101) flows through the midline, the two branches of the Urinary Bladder meridian (110, 110′) parallel to the Du meridian (101) at a distance of about 1.5 Cun (Cun is a measuring unit of distance in acupuncture). The two branches of the Gall Bladder meridian (111, 111′) line up with the two pupils and further parallel to the Urinary Bladder meridian (110, 110′) at a distance of about 0.75 Cun.

The Du meridian (101) starts from Changqiang (D1) acupoint near the tail bone, flows through the midline of the back and head, and ends at Yinjiao (D28) acupoints at the mouth area. The Du meridian (101) has 28 acupoints. Among them, Shenting (D24)(15), Shangxing (D23) (14), Xinhui (D22) (13), Qianding (D21) (12), Baihui (D20) (11), and Houding (D19) (10) are the acupoints within the Du meridian (101) located at the top of the head. In addition, an extra point located at the midpoint between the eyebrows and known as “Yingtang (80)” is on the Du meridian (101) between Shenting (D24) (15) and Suliao (D25) (FIG. 2).

The two branches of the Urinary Bladder meridian (110, 110′) are parallel to the Du meridian. They begin from the pair of Jingmin (B11) acupoints at the inner corners of the eyes, and end at the pair of Zhiyin (B167) acupoints at the outer side of the tips of the two little toes. Each branch of the Urinary Bladder meridian (110, 110′) contains 67 acupoints. The acupoint on one branch of the Urinary Bladder meridian are parallel or corresponding to the acupoints with the same name at the other branch of the Urinary Bladder meridian. For example, the acupoint of Zanhu (B12) (20) at one branch of the Urinary Bladder meridian (110) is parallel to Zanhu (B12) (20) of the other branch of the Urinary Bladder meridian (110′). The pair of Zanzhu (B12) (20, 20′), the pair of Meichong (B13) (21, 21′), the pair of Quchai (B14) (22, 22′), the pair of Wuchu (B15) (23, 23′), the pair of Chengguang (B16) (24, 24′), the pair of Tongtian (B17) (25, 25′), and the pair of Luoque (B18) (26, 26′) acupoints are the acupoints of the Urinary Bladder meridian that locate at the top of the head.

The two branches of the Gall Bladder meridian (111, 111′) are parallel to the Urinary Bladder meridian (110, 110′). They start from the pair of Tongziliao (Gb1) acupoints at the outer corners of the eyes, wind around the ears, flow through the sides of the body and the legs, and finally end at Foot-Qiaoyin (Gb44) acupoints at the outer sides of the tips of the fourth toes. Each branch of the Gall Bladder meridian (111, 111′) has 44 pairs of acupoints. Like the Urinary Bladder meridian, the acupoints on one branch of the Gall Bladder meridian are parallel to the acupoints with the same name at the other branch of the Gall Bladder meridian. The pair of Yangbai (Gb14) (30, 30′), Head-Lingqi (Gb15) (31, 31′), Muchuang (Gb16) (32, 32′), Zhengying (Gb17) (33, 33′), and Chengling (Gb18) (34, 34′) acupoints are the acupoints of the Gall Bladder meridian (110, 110′) that locate at the top of the head. In addition, the midpoints (70, 70′) of the upper eyelids are also shown in FIG. 2.

The study of acupuncture from the bioelectromagnetics aspect is a very recent development, as compared to the long history of traditional acupuncture. It is now known that the energies produced by the body generate an electrical field in and around the body. The resistance of acupoints ranges from 100-200 kV, while other areas of the skin have resistance up to 1 MV. Acupoints are about 50% more conductive than surrounding areas, and electric currents pass more preferentially when electrodes are placed on acupoints on the same meridian than on different meridians. Acupoints store charge to a greater degree than surrounding tissues and have lower resistance, having capacitance readings of 0.02-0.5 mF (microfarads) while non-points are measured at less than 0.01 mF. (Se Starwynn, D. Medical Acupuncture 2003; 13(1): article #7). In an electroacupuncturogram (EAG) study, Ionescu-Tirgoviste and Pruna concluded that meridians exist as canals in the interstitial spaces between the structures of the body along which endogenous electrical charges propagate. Blockages in the normal flow of these currents lead to high concentrations of positive or negative electrical charges that may cause pain and other symptoms of disease. They further characterized superficial skin acupoints as having: (a) high electric potentials (up to 300 mV); (b) high electric capacitance values (0.1-1 mF); (c) low electrical resistance; (d) increased skin respiration; (e) high local temperature; (f) spontaneous visible light emission from the Jing and Yuan points; and (g) sound signals at the acupuncture points (2-15 Hz, amplitude 0.5-1 mV, sharp or sine wave). The deep skin acupoints have: (a) low deep perception threshold to an electric stimulus; (b) capacitance (storage of electric charges); (c) electro-resonance with other acupoints; and (d) high conductivity of isotopic tracers. (See Starwynn, D. Medical Acupuncture, Id.).

In a microcurrent electroacupuncture study, the increases of skin electrical conductivity after electrical stimulation are found far greater in patients than in healthy people. Patients with pain and dysfunction have a build-up of electrical activity due to impaired circulation. The large increases in conductivity usually correlate with rapid symptomatic relief of pain and restricted range of motion. When, on the other hand, the conductivity readings do not change after stimulation, symptomatic results have often been less precise. The findings support the principle that large releases of cumulative ionic imbalances in the electrical systems of the body are associated with good clinical results. (See Starwyann, D. Medical Acupuncture Medical Acupuncture, Id.).

In general, acupuncture needles are made of metal. The needle punctures through the skin at the acupoints and effectively corrects the electromagnetic imbalance of the body, hence the therapeutic effects.

The objection of the present invention is to provide a non-invasive, safe, effective, convenient, and readily available device to afford a calming/soothing effect for persons suffering from unconsciousness, imbalanced state of mind. This calming/soothing effect improves sleep in persons suffering from sleep problems (e.g., difficulty in falling asleep, disturbed sleep, and/or insomnia); promotes relaxation in persons with stress/tension/anxiety; and regulates and maintains a relaxing and pleasant mood in persons with depression or unstable mood.

In one embodiment of the present invention, the acupressure device comprises a supporting member (1) and one metal member (3) attaching to the inner side (2) of the supporting member (1). A person in need of such calming/soothing effect wears the acupressure device so that the supporting member (1) is tightly secured around the head, and the metal member (3) is positioned at Baihui (D20) acupoint (11) of the head. When the supporting member (1) presses the metal member (3) against the Baihui (D20) acupoint (11), a tension is generated toward said Baihui (D20) acupoint (11) to send a signal that calms and sooths said human.

The supporting member (1) of the acupressure device is preferably made of a flexible or semi-flexible material such as cloth, SPANDEX fabric, plastic, rubber, or leather. In particular, various cloths, such as synthetic or natural fibers, or a blend of both, are preferred for making the supporting material. Useful fibers include cotton, Dacron, polyester, and nylon. Alternatively, semi-rigid or rigid material, such as a metal or a metal alloy, can be used as supporting material. Useful metals include iron, stainless steel, nickel, copper, tin, zinc, platinum, titanium, gold and silver, and the various alloys attainable with these metals.

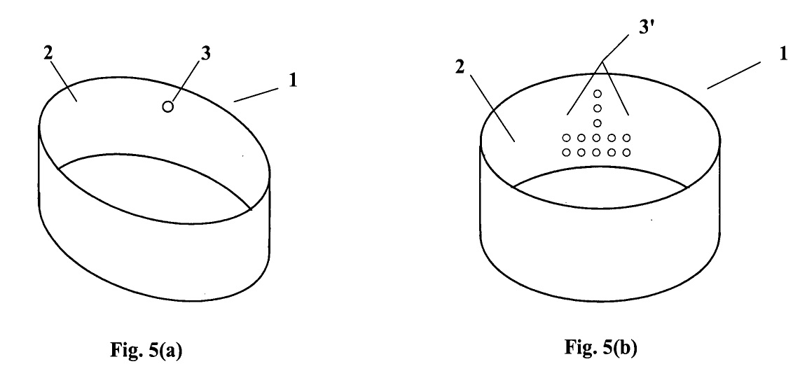

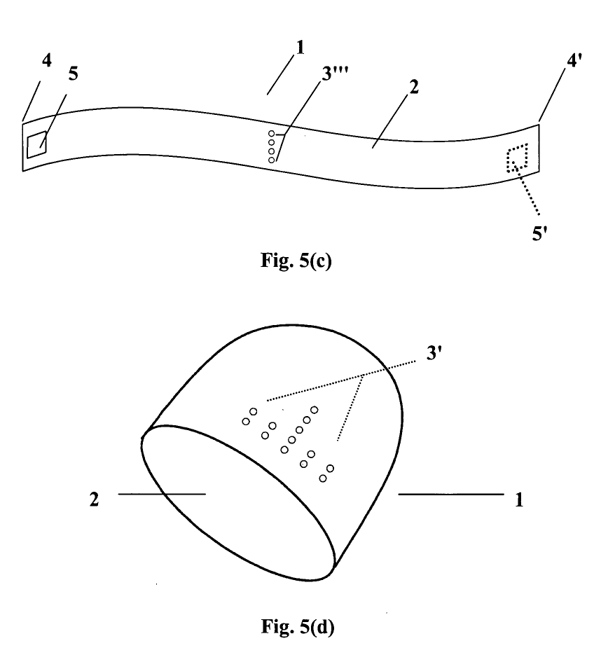

In one embodiment (FIG. 5(a)), the supporting member (1) can be in a form of headband. When worn on the head, the material of the supporting member (1) stretches and holds down the metal member (3) to exert pressure. In yet another embodiment (drawing not shown), the supporting member can be semi-rigid or rigid, such as a hair band, for adopting closely to the head whereupon the semi-rigidity or rigidity of the supporting member (1) holds down the metal member (3) to exerts pressure. In yet another embodiment, the supporting member (1) has a shape of a long strip with two opposite ends (4, 4′) (FIG. 5(c)). The supporting member (1) is tightened either by tying the ends (4, 4′) together or by fastening the ends (4, 4′) through fasteners (5, 5′) attached to the ends (4, 4′). Examples of the fasteners (5, 5′) includes, but are not limited to, hooks, pull-tabs, buckles, snap-hooks, adjuster bars, slides, cord locks, zipper pulls, modular buckles and VELCRO fasteners. In yet another embodiment (FIG. 5(d)), the supporting member (1) can be in a form of cap.

The metal member (3) is preferably made of electrically conductive metal. Because the metal member (3) is in contact with the skin directly or through the hairs and the skin is conductive, the imbalance of the electrical energy in the acupoint can be corrected through the contact with the metal member (3). Examples of the metal include, but are not limited to, gold, silver, platinum, copper, iron, stainless steel, nickel, aluminum, tin, titanium, and the alloys of these metals. The preferred metals are gold and silver. In addition, the use of magnetized metal is optional. The metal members have a smooth surface and can be in different shapes, including, but are not limited to, a bead, a rounded mass, a nubbin, a disk, a nipple, a nodule, a lump, a projection, a knob, or a seed, or any combination thereof. Optional a hole in the center or at the back of the metal member (3) is provided to facilitate the attachment of the metal member (3) to the supporting member (1). Alternatively, the metal member (3) of the present invention are attached chemically or mechanically to the inner side of the supporting member (1) by way of embedding in or affixing to the supporting member. One way to do so is by use of glue or adhesive. Another way to do so is by using prong(s) extended from the metal member (3). The prong(s) pass through the material of the supporting member (1) and is/are securely clamped at the other side of the supporting member (1). Yet another way to do so is by use of a thread to saw the metal member (3) onto the supporting member (1). The metal member can also be attached to the supporting member by way of molding from the supporting member, particularly if the supporting member is made of metal or plastic.

In another embodiment of the present invention, the acupressure device comprise a supporting member (1) and a plurality of metal members (3′) attaching to the inner side (2) of the supporting member (1). The metal members (3′) are positioned at a plurality of acupoints in a first treatment zone (201) on top of a human head (FIG. 2). One of the acupoints with the first treatment zone is Baihui (D20) acupoint (11). The first treatment zone includes a rectangular area extending from midpoints (70, 70′) of two upper eyelids to two Chengling(Gb18) acupoints (34, 34′) of the human head. Preferably, the metal members (3′) further positioned at at least one acupoint, which includes Yingtang acupoint (80), Shenting (D24) acupoint (15), Shangxing (D23) acupoint (14), Xinhui (D22) acupoint (13), Qianding (D21) acupoint (12), Houding (D19) acupoint (10), the pair of Zanzhu (B12) acupoints (20, 20′), the pair of Meichong (B13) acupoints (21, 21′), the pair of Quchai (B14) acupoints (22, 22′), the pair of Wuchu (B15) acupoints (23, 23′), the pair of Chengguang (B16) acupoints (24, 24′), the pair of Tongtian (B17) acupoints (25, 25′), the pair of Luoque (B18) acupoints (26, 26), the pair of Yangbai (Gb14) acupoints (30, 30′), the pair of Head-Lingqi (Gb15) acupoints (31, 31′), the pair of Muchuang (Gb16) acupoints (32, 32′), the pair of Zhengying (Gb17) acupoints (33, 33′), and the pair of Chengling (Gb18) acupoints (34, 34′).

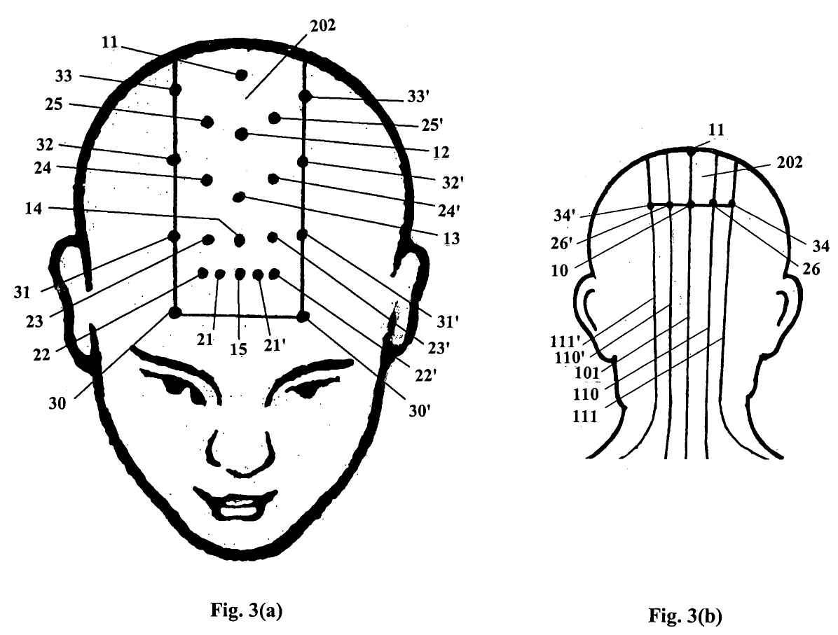

In yet another embodiment of the present invention, the acupressure device comprise a supporting member (1) and a plurality of metal members (3″) attaching to the inner side (2) of the supporting member. The metal members (3″) are positioned at a plurality of acupoints in a second treatment zone (202) on top of a human head (FIG. 3). One of the acupoints within the second treatment zone is Baihui (D20) acupoint (11). The second treatment zone includes a rectangular area extending from the two Yangbai (Gb14) acupoints (30, 30′) to the two Chengling (Gb18) acupoints (34, 34′). Preferably, the metal members (3″) are further positioned at at least one of the following acupoints: Shangxing (D24) acupoint (14), Xinhui (D23) acupoint (13), Qianding (D22) acupoint (12), the pair of Wuchu (B15) acupoints (23, 23′), the pair of Chengguang (B16) acupoints (24, 24′), the pair of Head-Lingqi (Gb15) acupoints (31, 31′), and the pair of Muchuang(Gb16) acupoints (32, 32′) within the second treatment zone of the human head.

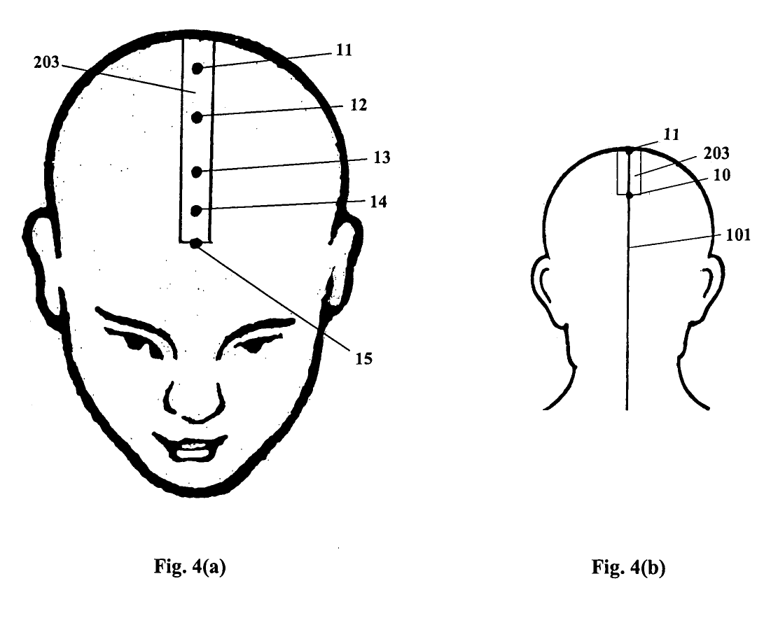

In yet another preferred embodiment of the present invention, the acupressure device comprises a supporting member (1) and a plurality of metal members (3′″) attaching to the inner side (2) of the supporting member (1). Preferably, the metal members (3′″) are further positioned at at least one of the following acupoints within the third treatment zone (203) on top of a human head (FIG. 4). The third treatment zone (203) is a rectangular area which is about 1 inch wide between Shenting (D24) acupoint (15) and Houding (D19) acupoint (10) of Du meridian (101). One of the multiple acupoints within the third treatment zone is Baihui (D20) acupoint (11).

When applying the acupressure device for producing a calming/soothing effect, the user first obtains an acupressure device of which the positional pattern of the metal members (3) is best fit to the user's need. The user wears the supporting member (1) in such a way that the metal members (3, 3′) are facing the wearer's head at the inner side of the supporting member (1). The supporting member (1) is then adjusted to a position where the metal members (3, 3′) are facing the acupoints of the head. The acupressure device can be worn from a few minutes to all day long to provide a calming/soothing effect in patients suffering from insomnia, stress/tension, and/or anxiety/depression.

The following examples are illustrative, but not limiting the scope of the present invention. Reasonable variations, such as those occur to reasonable artisan, can be made herein without departing from the scope of the present invention.

### EXAMPLE

### Preparation and Application of an Acupressure Device: Zzzz Wrap or Cap

Zzzz Wrap or Cap was prepared by attaching metal members to a band or a cap made of soft and stretchable synthetic cloth. The band was circular in shape and about 5 inches in width. The cap was in the shape of a head which covers the entire head. The metal members had prongs to pass through the fabric and secured to the cloth by folding down the prongs at the other side of the cloth. Five rows of metal members were aligned on the band or cap as shown in FIGS. 5(b) and 5(d), respectively. The first three rows each contains 1 bead. The fourth and fifth rows each contains 5 beads. The bead on each of the first three rows is in alignment with the bead at the center of each of the fourth and the fifth rows. The metal members were about 0.5-1 inch apart.

The user was instructed to wear Zzzz Wrap/Cap with the metal members in direct contact with the scalp with the fourth and fifth rows of metal members pressed close to the forehead and the first three rows of metal members positioned at the center of the head so that at least one of the beads can be pressed against Baihui (D20) acupoint (11) of the user.

### Case Reports

Case 1: Treatment of Anxiety and Insomnia

The patient was a 49-year old female. In 1996, the patient suffered from anxiety during the time period prior to obtaining her doctorate degree. She was temporarily treated with sedatives at that time.

In 2002, the patient again suffered from anxiety and insomnia after an automobile accident. The patient complained about sweating, nervousness, and nauseating sensation, requiring the treatment of sedatives.

In 2003, the patient was treated with needle acupuncture at the inventor's clinic. In between clinic visits, the patient wore Zzzz Wrap/Cap at night as instructed. The patient reported that she was able to sleep and the problems of anxiety and insomnia disappeared.

Case 2: Treatment of Anxiety, Insomnia, and Depression

The patient was a 64-year-old female who suffered from anxiety, depression and insomnia for more than 20 years. The patient had been treated with antidepressant and anti-anxiety medications, but the symptoms had not been controlled by the medications.

After treatment with needle acupuncture and Chinese herbal medications for 6 months, the patient was able to discontinue the antidepressants and anti-anxiety medications. During the time period between the treatments of acupuncture and herbal medicine, the patient wore Zzzz Wrap/Cap as instructed. Nine months later, the patient was able to stop all other treatments and just continued on the Zzzz Wrap/Cap. The patient reported stable mood during the day and sound sleep at night.

Case 3: Treatment of Insomnia

The patient was a 70-year-old female who has suffered from insomnia for more than 15 years and untreated with any medication.

The patient wore Zzzz Wrap/Cap during sleep as instructed. The patient reported improvement of sleep.

Case 4: Treatment of Insomnia

A 45-year-old female having difficulty in falling asleep was instructed to wear Zzzz Wrap/Cap when she went to sleep. The patient reported that she could fall asleep naturally and could sleep through the entire night until the next day.

While the invention has been described by way of examples and in term of the preferred embodiments, it is to be understood that the invention is not limited to the disclosed embodiments. On the contrary, it is intended to cover various modifications as would be apparent to those skilled in the art. Therefore, the scope of the appended claims should be accorded the broadest interpretation so as to encompass all such modifications.

1. An acupressure device comprising:
a supporting member (O), having an inner side (2); and
a metal member (3) attaching to the inner side (2) of said supporting member (1),
wherein said metal member (3) is positioned at Baihui (D20) acupoint (11) of a human located at top of a human head;
wherein when said supporting member (1) presses said metal member (3) against said Baihui (D20) acupoint (11) of said human head, a tension is generated toward said Baihui (D20) acupoint (11) to send a signal that calms and sooths said human.

2. The acupressure device according to claim 1, wherein said metal member (3) is an electrically conductive metal.

3. The acupressure device according to claim 2, wherein said electrically conductive metal is at least one selected from the group consisting of gold, silver, platinum, copper, iron, stainless steel, nickel, aluminum, tin, and titanium.

4. The acupressure device according to claim 1, wherein said metal member (3) is in the form of a bead, a rounded mass, a nubbin, a disk, a nipple, a nodule, a lump, a knob, a projection, a seed, a bump, a dome, or any combination thereof.

5. The acupressure device according to claim 1, wherein said supporting member (1) is made of at least one selected from the group consisting of cotton, Dacron, polyester, nylon, SPANDEX fabric, plastic, rubber, leather, and metal.

6. The acupressure device according to claim 5, wherein said supporting member (1) is in the form of a headband.

7. The acupressure device according to claim 5, wherein said supporting member (1) is in the form of a cap.

8. The acupressure device according to claim 7, wherein said supporting member (1) is a strip; wherein said strip has two opposite ends (4, 4′) at a longitudinal side of said strip; said two opposite ends (4, 4′) being able to be directly fastened together or fastened together through a pair of fasteners (5, 5′).

9. The acupressure device according to claim 8, wherein said fasteners (5, 5′) are selected from the group consisting of hooks, pull-tabs, buckles, snap-hooks, adjuster bars, slides, cord locks, zipper pulls, modular buckles and VELCROs; and wherein said fasteners are capable of being adjusted to tightness so as to exert pressure on said Baihui (D20) acupoint (11) on said human head.

10. A method for treating a human with insomnia comprising:
applying said acupressure device according to claim 1 to said human; wherein said metal member of said acupressure device is positioned at said Baihui acupoint (11) of said human head; whereby by tightening said acupressure device, said metal member exerts pressure to said Baihui (D20) acupoint (11).

11. A method for relieving stress/tension from a human comprising:
applying said acupressure device according to claim 1 to said human; wherein said metal member (3) of said acupressure device is positioned at said Baihui (D20) acupoint (11) of said human head; whereby by tightening said acupressure device, said metal member exerts pressure to said Baihui (D20) acupoint (11).

12. A method for reducing anxiety/depression from a human comprising;
applying said acupressure device according to claim 1 to said human; wherein said metal member (3) of said acupressure device is positioned at said Baihui (D20) acupoint (11) of said human head; whereby by tightening said acupressure device, said metal member exert pressure to said Baihui (D20) acupoint (11).

13. An acupressure device comprising:
a supporting member (1), having an inner side (2); and
a plurality of metal members (3′) attaching to said inner side of said supporting member,
wherein said plurality of said metal members (3′) are positioned at a plurality of acupoints in a first treatment zone (201) at top of a human head; wherein said first treatment zone includes a rectangular area extending from midpoints (70, 70′) of two upper eyelids to two Chengling (Gb18) acupoints (34, 34′) of said human head; one of said plurality of said acupoints being Baihui (D20) acupoint (11) of said head;
wherein when said supporting member (1) presses said plurality of said metal members (3′) against said Baihui (D20) acupoint (11) of said first treatment zone (201) on said human head, a tension is generated toward said first treatment zone to send a signal that calms and sooths said human.

14. The acupressure device according to claim 13, wherein said plurality of said metal members (3′) further positioned at at least one acupoint which is selected from the group consisting of Yingtang acupoint (80), Shenting (D24) acupoint (15), Shangxing (D23) acupoint (14), Xinhui (D22) acupoint (13), Qianding (D21) acupoint (12), Houding (D19) acupoint (10), Zanzhu (B12) acupoints (20, 20′), Meichong (B13) acupoints (21, 21′), Quchai (B14) acupoints (22, 22′), Wuchu (B15) points (23, 23′), Chengguang (B16) acupoints (24, 24′), Tongtian (B17) acupoints (25, 25′), Luoque (B18) acupoints (26, 26′), Yangbai (Gb14) acupoints (30, 30′), Head-Lingqi (Gb15) acupoints (31, 31′), Muchuang (Gb16) acupoints (32, 32′), Zhengying(Gb17) acupoints (33, 33′), and Chengling (Gb18) acupoints (34, 34′) of said head.

15. A method for treating insomnia in a human comprising:
applying said acupressure device according to claim 13 to said human; wherein said plurality of said metal members (3′) of said acupressure device is positioned at said plurality of said acupoints in said first treatment zone (201) of said human head; whereby by tightening said acupressure device, said plurality of said metal members (3′) exert pressure to said plurality of acupoints at said first treatment zone (201).

16. An acupressure device comprising:
a supporting member (1), having an inner side (2); and
a plurality of metal members (3″) attaching to said inner side of said supporting member (1);
wherein said plurality of said metal members (3″) are positioned at a plurality of acupoints at a second treatment zone (202) at top of a human head; wherein said second treatment zone (202) includes a rectangular area extending from two Yangbai (Gb14) acupoints (30, 30′) to two Chengling (Gb18) acupoints (34, 34′) of said head; one of said plurality of said acupoints being Baihui (D20) acupoint (11);
wherein when said supporting member (1) presses said plurality of said metal member (3″) against said plurality of said acupoints at said second treatment zone (202) of said human head, a tension is generated toward said plurality of said acupoints at said second treatment zone (202) to send a signal that calms and sooths said human.

17. The acupressure device according to claim 16, wherein said plurality of said multiple metal members (3″) are further positioned at at least one acupoint which is selected from the group consisting of Shangxing (D23) acupoint (14), Xinhui (D22) acupoint (13), Qianding (D21) acupoint (12), Wuchu (B15) acupoints (23, 23′), Chengguang (B16)acupoints (24, 24′), Head-Lingqi (Gb15) acupoints (31, 31′) and Muchuang (Gb16) acupoints (32, 32′) of said human head.

18. A method for treating insomnia in a human comprising:
applying said acupressure device according to claim 15 to said human; wherein said plurality of said metal member (3″) of said acupressure device is positioned at at least one of said acupoints at said second treatment zone (202) of said human head; whereby by tightening said acupressure device, said plurality of said metal members exert pressure to said second treatment zone (202).

19. An acupressure device comprising:
a supporting member (1), having an inner side (2); and
a plurality of metal members (3′″) attaching to said inner side (2) of said supporting member (1);
wherein said plurality of said metal members (3′″) are positioned at a plurality of acupoints at a third treatment zone (203) at top of a human head; wherein said third treatment zone (203) is a rectangular area which is about 1 inch wide between Shenting (D24) acupoint (15) and Houding (D19) acupoint (10) of Du meridian (101); one of said plurality of said acupoints being Baihui (D20) acupoint (11);
wherein when said supporting member (1) presses said plurality of said metal member (3′″) against said third treatment zone (203) of said human head, a tension is generated toward said plurality of said acupoints at said third treatment zone (203) to send a signal that calms and sooths said human.

20. A method for treating insomnia in human comprising:
applying said acupressure device according to claim 19 to said human; wherein said plurality of said metal members (3′″) of said acupressure device is positioned at at least one of said acupoints at said third treatment zone (203) of said human head; whereby by tightening said acupressure device, said plurality of said metal members (3′″) exert pressure to said plurality of acupoints at said third treatment zone (203).
